(19) United States
(12) Patent Application Publication
(10) Pub. No.: US 2024/0370999 A1
(43) Pub. Date: Nov. 7, 2024

(54) METHOD OF ADJUDICATING AN IMAGED LESION

(71) Applicant: Siemens Healthineers AG, Forchheim (DE)

(72) Inventors: Philipp HOELZER, Tokyo (JP); Tobias HECKEL, Muenchen (DE); Stefan ASSMANN, Buckenhof (DE); Ayse KARABAYIR, Roethenbach (DE); Torbjoern KLATT, Erftstadt (DE); Robin GUTSCHE, Erlangen (DE); Sebastian SCHMIDT, Weisendorf (DE); Ali KAMEN, Skillman, NJ (US); Vivek SINGH, Princeton, NJ (US); Alexander BROST, Erlangen (DE); Matthias SIEBERT, Marloffstein (DE); Jonathan SPERL, Bamberg (DE)

(73) Assignee: Siemens Healthineers AG, Forchheim (DE)

(21) Appl. No.: 18/652,286

(22) Filed: May 1, 2024

(30) Foreign Application Priority Data
May 4, 2023 (EP) 23171524.4

(51) Int. Cl. G06T 7/00

(52) U.S. Cl. CPC G06T 7/0012; G06T 2207/10081; G06T 2207/20081; G06T 2207/30204

(57) ABSTRACT

A computer-implemented method of adjudicating an imaged lesion, comprising: receiving a diagnostic image showing a lesion; processing the diagnostic image in a machine learning algorithm previously trained to classify the lesion and to propose, based on a lesion class for the lesion, a blood test panel suited to adjudicate the lesion; and outputting the proposed blood test panel to a user.

### CROSS-REFERENCE TO RELATED APPLICATION(S)

The present application claims priority under 35 U.S.C. § 119 to European Patent Application No. 23171524.4, filed May 4, 2023, the entire contents of which are incorporated herein by reference.

### BACKGROUND

Various medical conditions can be detected using diagnostic imaging techniques. Particularly in the case of an imaged lesion indicating the presence of a tumor, it is important to be able to correctly assess the nature of the lesion. A false negative diagnosis results in the medical condition going undetected. A false positive diagnosis can be followed by unnecessary and costly invasive procedures. It is known to apply machine learning to assist in identifying imaged lesions, for example a neural network can be trained on many CT images to learn to classify a lesion, primarily on the basis of shape and size. However, the use of the known machine learning methods is largely limited to classification. Therefore, in order to adjudicate a lesion, i.e. to reach a decision as regards the degree of malignancy of the lesion, a clinician must rely on experience and/or avail of further diagnostic tests.

Therefore, in addition to diagnostic imaging, the assessment of a medical condition preferably involves at least one further laboratory diagnostic test. Diagnostic imaging and laboratory diagnostics are fundamentally different in the way they query underlying information. This can be used to good effect in the context of complex clinical questions such as assessing the malignancy of a tumor or lesion, helping a clinician to plan a suitable schedule for subsequent patient management. For example, if a diagnostic image and a blood test both independently suggest malignancy, an invasive procedure such as biopsy or resection can be justified with a high degree of confidence.

However, there is a vast number of established and proprietary blood tests, each configured to detect a certain combination of biological markers present above certain threshold levels in the blood (biological markers present in the blood are generally referred to simply as “blood markers”). Blood tests can differ in their range of validity across various subgroups such as lesion size range, histopathological subtype, patient age, patient smoking history, nodule composition, disease prevalence etc. Blood tests can also differ with regard to the clinical question, i.e. whether the lesion is benign or malignant, whether the lesion is a primary or secondary tumor, whether the lesion is aggressive or indolent, the level of invasiveness of the lesion, whether the lesion is a small-cell or non-small-cell carcinoma, whether the lesion shows druggable molecular alterations, etc. Clearly, it is not practicable to apply every test for every patient.

### SUMMARY

It is therefore an object of one or more embodiments of the present invention to provide an improved way of combining a diagnostic imaging procedure and a laboratory diagnostic procedure.

At least this object is achieved by a method of adjudicating an imaged lesion and by the claimed machine learning algorithm according to one or more embodiments of the present invention.

According to an embodiment of the present invention, the computer-implemented method for use in adjudicating an imaged lesion comprises steps of: receiving a diagnostic image showing a lesion; processing the diagnostic image in a machine learning algorithm trained to assess or classify an imaged lesion and trained to propose, on the basis of lesion class, a blood test panel comprising markers most suited to adjudicate the imaged lesion; and outputting the proposed blood test panel to a user.

In the context of embodiments of the present invention, the proposed blood test panel may represent a single blood test or several blood tests. A clinician can then order one or more blood samples to be obtained and processed according to the proposed blood test panel, and can then adjudicate the lesion from the blood test results. An advantage of the inventive computer-implemented method is that an analysis of image-based features is sufficient for the machine learning algorithm to recommend the most promising blood test panel for that patient in order to reliably adjudicate the lesion, i.e. to answer a specific clinical question such as the degree of malignancy of the lesion. After using the inventive computer-implemented method, it can be sufficient to carry out a single blood test on a panel comprising the set of blood biomarkers that will provide a most informative result to help the clinician obtain an answer to a specific clinical question. This is an improvement over known procedures in which several successive blood tests may be required in order to facilitate satisfactory adjudication of a lesion.

Typically, a lesion is classified as low risk, intermediate risk, or high risk in the context of its malignancy. Depending on the risk group, subsequent patient management can involve surveillance imaging, biopsy, surgery, radiation therapy, ablation etc. as appropriate. However, if a lesion is unambiguously classified in a risk group, ordering of a subsequent blood test is of little clinical benefit. This might be the case for a very large lesion (greater than 30 mm). The clinical impact of a blood test is highest if an imaged lesion cannot be assigned unequivocally to a risk group, i.e. the malignancy risk of the lesion is at the threshold between low risk and intermediate risk, or at the threshold between intermediate risk and high risk. In such threshold cases, the machine learning algorithm can choose blood markers on the basis of their estimated effect size.

In the claimed computer-implemented method, the machine learning algorithm preferably disregards any blood marker with a small effect size and selects only blood markers with large effect sizes. In this way, the machine learning algorithm can propose a blood test panel that is most useful in identifying the correct risk group into which the lesion can be assigned, i.e. the machine learning algorithm provides a way of more accurately adjudicating a lesion.

The machine learning algorithm for use in the inventive computer-implemented method is preferably a multi-class classifier configured to classify an imaged lesion into one of a plurality of predefined classes; and to identify a blood panel which will deliver the most accurate results in a subsequent step of adjudicating the imaged lesion.

Training such a machine learning algorithm preferably comprises the steps of:
A) compiling a training data set comprising an image showing a lesion, a classification of the lesion, and the patient outcome;
B) assigning a ground truth to the training data set, which ground truth comprises a set of biomarkers of a blood panel performed for the respective patient and the blood panel result;
C) applying the machine learning algorithm to the training data set to identify a set of blood panel biomarkers approximating the ground truth; and repeating steps A-C for a plurality of training data set until a desired level of accuracy has been achieved.

An object of one or more embodiments of the present invention is also achieved by a non-transitory computer program product with a computer program that is directly loadable into the memory of a data-processing apparatus, and which comprises program units to perform the steps of the inventive computer-implemented method when the program is executed by the control unit.

The machine learning algorithm can be realized as a module of a computer program which can run on any suitable platform and which comprises program units to perform the steps of the inventive computer-implemented method to classify an imaged lesion, to identify blood markers suited to adjudicate the imaged lesion, and to output a blood test panel comprising the identified blood markers. In the context of embodiments of the present invention, a computer program that incorporates the trained machine learning algorithm and has a mechanism, device, apparatus and/or means of/for receiving relevant input (a diagnostic image and any other relevant inputs as described above can be input to the computer program in any suitable file format) and a mechanism, device, apparatus and/or means of/for outputting the proposed blood panel is referred to herein as an “adjudication program” or “adjudication assistant”. The computer program can be configured to receive or read a diagnostic image (and any other relevant inputs) in any suitable format and can present the proposed blood test panel to the user in any suitable manner (e.g. to print the result and/or to send it to a further device such as a computer, a mobile device, a central laboratory, etc.). The inventive computer-implemented method can be used by a clinician to assist in stratifying patients into different risk groups with corresponding diagnostic workup.

Particularly advantageous embodiments and features of the present invention are given by the dependent claims, as revealed in the following description. Features of different claim categories may be combined as appropriate to give further embodiments not described herein.

In the context of embodiments of the present invention, expressions such as “machine learning algorithm”, “neural network”, “artificial intelligence tool” and “AI model” shall be understood to be synonyms and may be used interchangeably. The terms “blood test panel”, “blood panel” and “test panel” may be used interchangeably.

The imaging modality used to obtain the diagnostic image can be computed tomography (CT), magnetic resonance imaging (MRI), positron emission tomography (PET), single-photon emission computed tomography (SPECT), X-ray imaging, ultrasound imaging, optical coherence tomography (OCT), photoacoustic imaging, etc. In the following, without restricting the present invention in any way, it may be assumed that the diagnostic image is a computed tomography image (in the form of a 3D volume or a 2D slice), for example an image can be a process spectrally resolved CT image, a photon-counting CT image; a high-resolution CT image, a contrast-enhanced CT image, a pair of inspiratory and expiratory scans, a gated CT scan, a stationary multi-source CT scan.

The inventive computer-implemented method is applicable to various kinds of lesion, for example the imaged lesion can be any of: a pulmonary lesion, an adrenal lesion, a renal lesion, a hepatic lesion, a pancreatic lesion, a mammary lesion. Any of these can be a tumor, and it is important to reliably determine whether the lesion is benign or malignant. If the lesion is malignant, it is important to reliably assess the degree of malignancy. One or more embodiments of the present invention are particularly suited to assist in adjudicating lesions such as pulmonary nodules, since these can be difficult for a clinician to classify from visual assessment of a diagnostic image, and it can be difficult for a clinician to choose suitable biomarkers to assist in adjudicating an imaged pulmonary nodule.

The machine learning algorithm or “AI model” may include an artificial neural network to apply deep learning techniques. The machine learning algorithm is trained to propose a suitable blood test panel to most accurately adjudicate an imaged lesion. The machine learning algorithm can be implemented as a multi-task classifier which first classifies the imaged lesion into one of several predefined classes, and then identifies the blood test panel that will be best at adjudicating the lesion.

In the following, the machine learning algorithm may be assumed to comprise a neural network such as a convolutional neural network (CNN). Any suitable CNN architecture may be used, for example AlexNet. A supervised training procedure of such a neural network preferably comprises a technique of backward-propagation to find the best set of parameters that will enable the network to infer a ground truth from a diagnostic image, in this case to infer the lesion class of an imaged lesion in its first task, and to infer the most suitable blood test panel in its second task. The training procedure is preferably configured to achieve a favourably low cost function.

The lesion classification stage of the machine learning algorithm preferably influences the internal configuration of the proposed blood test panel, i.e. the operating point along the receiver operating characteristic curve (ROC curve) of the entire test panel, the decision thresholds of individual blood markers, the weights of the individual blood markers, etc.

In addition to the image, the associated blood test for that patient, and the patient outcome (e.g. development of lung cancer and the elapsed time to diagnosis), a training data set preferably also comprises annotated image data. The size, shape and position of a lesion—along with various other factors—are highly relevant to the question of its malignancy. Therefore, in a preferred embodiment of the present invention, the training method comprises a step of annotating an image of a training dataset to identify features including any of: lesion size, lesion quantity, lesion composition, lesion location, lesion shape, lesion density, texture features, semantic features, vascular convergence, cavitation, calcification.

A training data set is preferably also augmented by the occurrence and extent of comorbidities such as emphysema, fibrosis, edema, consolidation, opacities, pleural effusion, atelectasis, pulmonary emboli, etc. Further relevant information can be the patient's subcutaneous and visceral fat content, bone mineral density, muscle density, organ volumes, etc. A training data set is preferably also augmented by relevant clinical factors such as patient age, sex, smoking history, history of cancer, familial cancer predisposition, occupation, etc.

Some blood markers are detectable in the bloodstream only if the tumor sheds sufficient material in the first place. For example, freely circulating tumor DNA (ctDNA) is released into the bloodstream through apoptotic or necrotic processes, and predominately in the case of larger tumor sizes. Equally, other systemic markers can show higher diagnostic accuracy for smaller nodule sizes, for example autoantibodies and/or micro-RNA (miRNA). The machine learning algorithm learns to identify which markers are most suitable for including in a blood test panel to adjudicate a lesion on the basis of its class.

The machine learning algorithm is trained to compile a blood test panel comprising any markers such as: carcinoembryonic antigen (CEA), pro-gastrin-releasing-peptide (ProGRP), cytokeratins such as Cyfra 21-1, SCC, neuron specific enolase (NSE), C-reactive protein (CRP), interleukin-6 (IL-6), serum amyloid A1 (SAA1), circulating tumor DNA (ctDNA) (mutations and genetic alterations, methylation patterns, fragmentation patterns); circulating tumor cells (chromosome abnormalities); proteins (autoantibodies, oncofetal antigens, structural proteins, hormones, glycoproteins, storage proteins, enzymes), circulating RNA, micro-RNA (miRNA), metabolites, RNA, sugars, exosomes, extracellular vesicles, etc.

The blood markers are chosen depending on the class to which the lesion has been assigned as well as other factors such as its size. For example, certain blood markers have different sensitivities, so that, if the size of the imaged lesion suggests that it might be outside or near of the range of validity of a certain marker, that marker can be assigned a lower weight in the blood panel. Depending how well the shape characteristics of a lesion correlate with the accuracy of a blood marker, the weight of such can be set accordingly.

The machine learning algorithm preferably applies imaging characteristics such as spiculation and nodule type to configure detection thresholds and risk score weighting factors of markers of a fixed blood test panel. A fixed blood test panel can be a proprietary panel or can have been compiled by a laboratory or a clinician.

For example, a diagnostic image could show a pulmonary nodule located in the periphery of the lung, with solid appearance, homogeneous density and without spiculation: these features are indicative of small cell lung cancer (SCLC). The machine learning algorithm preferably specifies a high threshold for the SCC biomarker in the subsequent laboratory processing step, thus according a relatively high weight to a negative SCC test result (this is because SCLC must have low SCC values). In this way, the sensitivity of the test is increased for SCLC. Since SCLC is the most aggressive form of lung cancer, a high specificity of diagnostic imaging and laboratory blood testing would then accurately indicate a high probability of malignancy for the imaged lesion. The physician and patient can be assured that a subsequent invasive and costly medical procedure is justified.

Since the machine learning algorithm is to be trained to select a set of blood markers that will assist in adjudicating a lesion, each training data set also includes a blood panel and laboratory test results carried out for that patient. For the training data set, a blood panel need not have been scheduled in connection with an imaged lesion, and it is sufficient that the blood test was performed after detection of the lesion by diagnostic imaging. Preferably, each training data set also includes the patient outcome, e.g. whether or not the imaged lesion led to cancer.

The accuracy of a machine learning algorithm depends to a large extent on the number of datasets with which it is trained. In a preferred embodiment of the present invention, the machine learning algorithm is trained using at least several hundred training data sets, more preferably several thousand training data sets. Images used to obtain the machine learning algorithm can be obtained from diagnostic imaging modalities used in one or more clinics. Equally, the machine learning algorithm could also be trained with synthetically generated images in order to improve its accuracy in classifying lesions.

The trained machine learning algorithm can then be used by a clinician to assist in adjudicating an imaged lesion. With the inventive computer-implemented method, a suitable diagnostic image is obtained and input to the machine learning algorithm, which returns a proposed blood test panel comprising a set of blood markers chosen to deliver the most conclusive results.

Of course, the machine learning algorithm can continue to learn by including the actual patient outcome some time after adjudication of a lesion using a blood test panel proposed by the machine learning algorithm.

In a preferred embodiment of the present invention, the machine learning algorithm may also propose a suitable laboratory technique for analysis of the proposed blood panel. A laboratory analysis technique can be any of: a next-generation sequencing technique, a polymerase chain reaction technique (PCR), a mass spectrometry technique, an immunoassay technique, a fluorescence in-situ hybridization (FISH) technique, an electrochemical sensing technique.

However, even with accurate classification of the lesion and choice of suitable blood markers, the machine learning algorithm may foresee that the proposed blood panel—even though it comprises the most suitable markers—may return an inconclusive result. Therefore, the machine learning algorithm is preferably also trained to also identify one or more further biomarkers for an additional laboratory test. For example, should the machine learning algorithm predict an inconclusive blood panel result, it may propose one or more further biological markers that may then be helpful in adjudicating the classified lesion. Such biological markers may be obtained from a nasal swab, urine, a bronchial swab, saliva, breast milk, etc.

The inventive adjudication program can run on a processing unit connected to an imaging modality in a clinic. Equally, such a computer program can run on a remote server or cloud computing arrangement.

In a preferred embodiment of the present invention, the adjudication program comprises a software module configured to automatically order the recommended blood test. In a further preferred embodiment of the present invention, the adjudication program comprises a FHIR (Fast Healthcare Interoperability Resources) communication module configured to pre-fetch clinical data from an electronic medical record of the patient as additional input to the machine learning algorithm. In a further preferred embodiment of the present invention, the machine learning algorithm is configured to recommend several blood tests to be performed at different stages to facilitate a long-term trend analysis. This can be especially useful if lesion classification is at the threshold between two risk groups.

### BRIEF DESCRIPTION OF THE DRAWINGS

Other objects and features of the present invention will become apparent from the following detailed descriptions considered in conjunction with the accompanying drawings. It is to be understood, however, that the drawings are designed solely for the purposes of illustration and not as a definition of the limits of the present invention.

FIG. 1 illustrates the inventive computer-implemented method of adjudicating an imaged lesion;

FIG. 2 illustrates a further aspect of the inventive computer-implemented method;

FIG. 3 illustrates an exemplary relationship between lesion size and the diagnostic accuracy of biomarkers;

FIG. 4 illustrates a further aspect of using biomarkers to adjudicate a lesion;

FIG. 5 illustrates a training stage of the machine learning algorithm used by the inventive computer-implemented method;

FIG. 6 illustrates an exemplary embodiment of the present invention;

FIG. 7 shows a conventional approach in adjudicating an imaged lesion.

### DETAILED DESCRIPTION

In the diagrams, like numbers refer to like objects throughout. Objects in the diagrams are not necessarily drawn to scale.

In the following diagrams, the diagnostic image is represented by a pulmonary CT image, and the lesion is represented by a pulmonary nodule. However, as indicated above, the inventive computer-implemented method is applicable to various kinds of lesion, and the images and lesions shown in the drawings are merely exemplary.

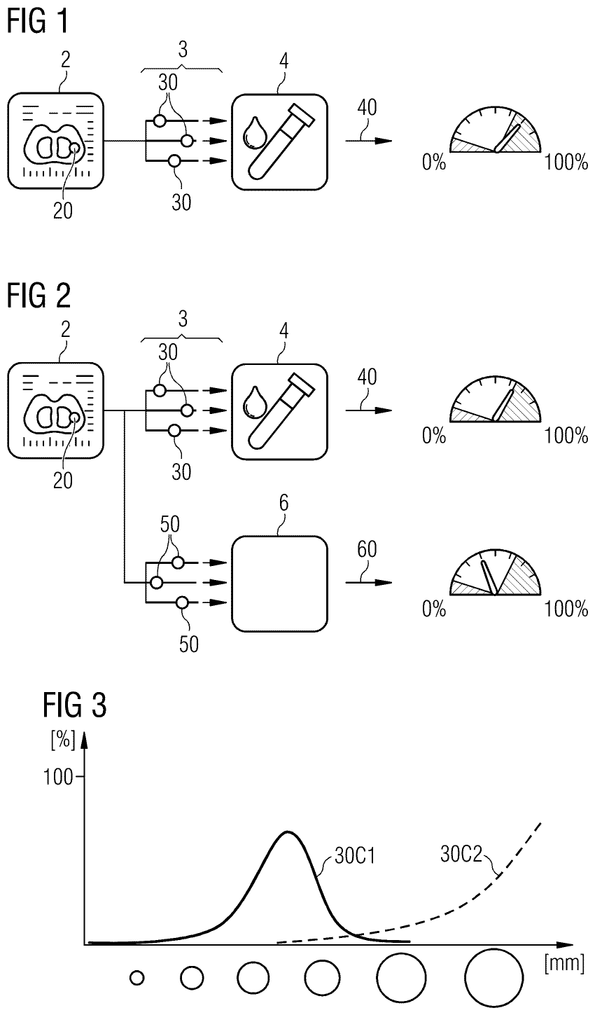

FIG. 1 illustrates the inventive computer-implemented method. An image 2 is obtained for a patient, showing a pulmonary nodule 20. The image 2 is processed by the machine learning algorithm 1NN, which outputs a blood test panel 3 comprising the set of blood markers 30 that is most likely to return a conclusive result regarding the malignancy (or not) of the imaged lesion.

A clinician can then order a blood sample to be obtained and sent to a laboratory for processing. The blood sample can be processed according to the blood test panel 3 using any suitable technique such as next-generation sequencing, polymerase chain reaction (PCR), mass spectrometry, immunosorbent assay (ELISA), enzyme-linked fluorescence in-situ hybridization (FISH), etc. The machine learning algorithm 1NN may also recommend a different processing technique as appropriate.

On the right-hand side of the diagram, a dial represents a scale between 0% (lowest risk of malignancy, i.e. the imaged tumor is most likely benign) and 100% (highest risk of malignancy). The machine learning algorithm will have compiled a blood panel which, after laboratory processing of the blood sample 4, will return information allowing the clinician to accurately determine the malignancy of the lesion. The outcome of the adjudication is represented symbolically by the dial indicator. In this example, the indicator is clearly positioned within one of three risk groups.

FIG. 2 illustrates a further aspect of the inventive computer-implemented method. Here, the machine learning algorithm 1NN predicts that the proposed blood panel 3 may be inconclusive, and has identified one or more further biomarkers 50 for an additional laboratory test 6. The diagram shows that the results 40 of the blood sample 4 tested according to the blood panel 3 are indeed inconclusive in this case (the “dial indicator” is positioned at a threshold between two risk groups), and that the additional laboratory test 6 delivers a conclusive result 60. In this exemplary embodiment, the lower “dial indicator” is clearly positioned within the intermediate risk group, i.e. the lesion can be reliably adjudicated and the clinician can decide on subsequent procedure with more confidence. More importantly, the patient need not—at this stage—undergo an unnecessary and costly invasive procedure more appropriate to a high-risk tumor.

FIG. 3 illustrates an exemplary relationship between lesion size (X-axis) and the diagnostic accuracy (Y-axis) of two exemplary biomarkers. The diagram shows that the diagnostic accuracy curve 30C1 of a first biomarker is highest for mid-size lesions and low for small lesions and large lesions, while the diagnostic accuracy curve 30C2 of a second biomarker is high only for large lesions. The machine learning algorithm applies this type of information when putting together a diagnostic blood panel 3 for an imaged lesion 20.

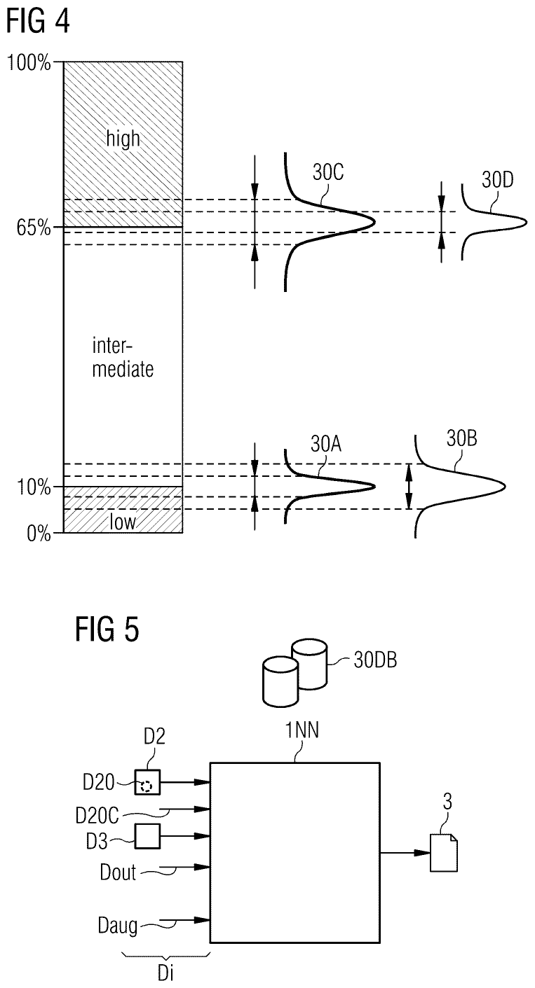

FIG. 4 illustrates a further aspect of using biomarkers to adjudicate a lesion. The vertical axis represents a pre-test probability of malignancy. Representing an exemplary situation, the diagram can be understood as follows: if the likelihood of a lesion being malignant is 65% or more, a biopsy or similar is justified; if the likelihood of a lesion being malignant is below 65%, the clinician may order a PET-CT or other follow-up CT and/or a less invasive procedure such as a percutaneous biopsy); if the likelihood of a lesion being malignant is less than 10%, the clinician may opt for surveillance of the patient. However, not all biomarkers are suited to obtaining a conclusive probability of malignancy. For example, biomarker 30A (represented by a diagnostic accuracy curve) is unsuited for adjudicating between a low probability of malignancy and an intermediate probability of malignancy (its effect size is small), while biomarker 30B is better suited (its effect size is large); similarly, biomarker 30C is suited for adjudicating between a low probability of malignancy and an intermediate probability of malignancy, while biomarker 30D is unsuited. The machine learning algorithm 1NN applies this type of information when putting together a set of biomarkers 30 for a diagnostic blood panel 3.

FIG. 5 illustrates a training stage of the machine learning algorithm 1NN. Here, training data sets are input to the machine learning algorithm 1NN. Each training data set Di comprises an image D2 showing a lesion D20, the lesion class D20C, the blood panel D3 performed for that patient, and the established malignancy of the imaged lesion, i.e. the patient outcome Dout. Each dataset Di can be augmented with other relevant information Daug such as co-morbidities of the patient, the patient's EHR, etc.

The machine learning algorithm 1NN is trained with at least 100 such training data sets Di. More preferably however, the machine learning algorithm 1NN is trained with several thousand training data sets Di. Training is deemed complete when the machine learning algorithm 1NN is able to classify an imaged lesion with a high degree of accuracy and is able to select a set of most suitable blood markers from an extensive blood marker database 30DB, presenting this as a proposed blood panel 3 to assist in reliably adjudicating the imaged lesion.

The blood marker database 330DB is not restricted to the blood markers appearing in the blood panels D3 of the training data sets Di, but can include further blood markers also, for example, markers chosen on the basis of patient outcome data, biopsy data or follow-up imaging data.

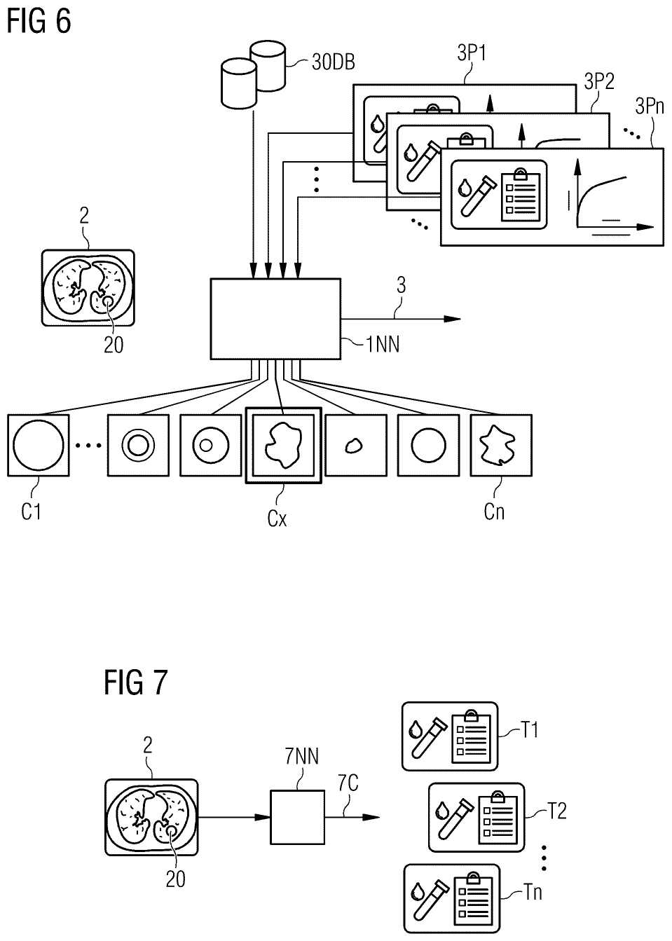

FIG. 6 illustrates an exemplary embodiment of the present invention. Here, an image 2 showing a lesion 20 is input to the machine learning algorithm 1NN. In a first stage, the machine learning algorithm 1NN classifies the lesion 20 into one of a predetermined set of lesion classes C1, . . . , Cn. In this example, the machine learning algorithm 1NN has classified the lesion 20 as belonging to class Cx.

In a subsequent stage, the machine learning algorithm 1NN identifies a blood test panel (with one or more blood markers) that will return the most conclusive information regarding the lesion 20. The machine learning algorithm 1NN can compile a blood panel from a plurality of individual blood markers whose specifics are stored in a database 30DB. In this exemplary embodiment, the machine learning algorithm 1NN can also choose from several predefined blood panels 3P1, . . . , 3Pn (e.g. proprietary blood panels), each with a previously established set of blood markers and linked to a predefined diagnostic technique. In a final step, the machine learning algorithm 1NN outputs the proposed blood panel 3 to a user.

FIG. 7 shows a conventional approach. Here, a pulmonary CT image 2 is obtained for a patient, showing a pulmonary nodule 20. The image 2 is processed by a machine learning algorithm 7NN which has been trained to classify the lesion. For some types and size of lesion, classification can be done to a high degree of accuracy, allowing a clinician to adjudicate the lesion 20 as benign or malignant with some degree confidence, based on the lesion class 7C returned by the machine learning algorithm 7NN. However, not all lesions can be reliably adjudicated. In an attempt to obtain a reliable diagnosis for the patient, the clinician may then order blood tests T1, . . . , Tn on various different biomarkers. Several tests are generally required, because the known blood tests generally have either a high negative predictive or a high positive predictive value, but rarely both. A single blood test chosen on the basis of the lesion class 7C determined using the prior art approach is therefore usually only of limited use in the adjudication of a lesion. The results of a single blood test are often inconclusive, and the clinician may resort to an invasive procedure in order to obtain certainty for the patient. This can add significantly to the overall cost of treatment and can cause undue stress to the patient.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The non-transitory computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for use in adjudicating an imaged lesion, the computer-implemented method comprising:
obtaining a diagnostic image showing a lesion;
inputting the diagnostic image to a machine learning algorithm previously trained to classify the lesion and to propose, based on a lesion class for the lesion, a blood test panel including markers suited to adjudicate the lesion; and
outputting the blood test panel to a user.

2. The computer-implemented method according to claim 1, wherein the blood test panel comprises markers including at least one of: circulating tumor DNA, circulating tumor cells, proteins, circulating RNA, metabolites, sugars, or exosomes.

3. The computer-implemented method according to claim 1, wherein the machine learning algorithm is trained to estimate a malignancy risk of the lesion, and to include at least one marker with a large effect size in the blood test panel when the malignancy risk is at a threshold between two risk groups.

4. The computer-implemented method according to claim 1, wherein the blood test panel includes one or more of: an operating point along a receiver operating characteristic curve of the blood test panel, a decision threshold of a blood marker, or a weight of a blood marker.

5. The computer-implemented method according to claim 1, wherein the machine learning algorithm is trained to identify at least one laboratory analysis technique for analysis of the blood test panel.

6. The computer-implemented method according to claim 5, wherein the at least one laboratory analysis technique is: a next generation sequencing technique, a polymerase chain reaction technique, a mass spectrometry technique, an immunoassay technique, a fluorescence in-situ hybridization technique, or an electrochemical sensing technique.

7. The computer-implemented method according to claim 1, wherein the machine learning algorithm is trained to identify a number of further biological markers to assist in adjudicating the lesion.

8. The computer-implemented method according to claim 7, wherein a further biological marker is obtainable from a nasal swab, urine, a bronchial swab, or saliva.

9. The computer-implemented method according to claim 1, wherein the diagnostic image is a computed tomography image.

10. The computer-implemented method according to claim 1, wherein the lesion is a pulmonary lesion, an adrenal lesion, a renal lesion, a hepatic lesion, a pancreatic lesion, or a mammary lesion.

11. The computer-implemented method according to claim 1, wherein the machine learning algorithm is a two-step classifier configured to
classify the lesion into one of a plurality of defined classes,
identify a number of blood markers suited to adjudicate the lesion, and
output the blood test panel including the blood markers.

12. A method of adjudicating an imaged lesion, the method comprising:
obtaining a diagnostic image showing a lesion;
processing the diagnostic image using the computer-implemented method according to claim 1 and receiving the blood test panel; and
obtaining a blood sample and performing laboratory processing of the blood sample according to the blood test panel.

13. A data processing apparatus configured to perform the computer-implemented method according to claim 1, wherein the data processing apparatus comprises:
an input interface configured to obtain the diagnostic image;
the machine learning algorithm previously trained to classify the lesion and to specify, based on the lesion class, the blood test panel suited to adjudicate the lesion; and
an output device configured to output the blood test panel to the user.

14. A non-transitory computer program product comprising instructions which, when executed by a computer, cause the computer to carry out the computer-implemented method according to claim 1.

15. A non-transitory computer-readable storage medium storing computer-executable instructions that, when executed at a computer, cause the computer to carry out the computer-implemented method of claim 1.

16. The computer-implemented method according to claim 2, wherein the machine learning algorithm is trained to estimate a malignancy risk of the lesion, and to include at least one marker with a large effect size in the blood test panel when the malignancy risk is at a threshold between two risk groups.

17. The computer-implemented method according to claim 16, wherein the machine learning algorithm is trained to identify at least one laboratory analysis technique for analysis of the blood test panel.

18. The computer-implemented method according to claim 16, wherein the machine learning algorithm is trained to identify a number of further biological markers to assist in adjudicating the lesion.

19. The computer-implemented method according to claim 2, wherein the machine learning algorithm is trained to identify at least one laboratory analysis technique for analysis of the blood test panel.

20. The computer-implemented method according to claim 19, wherein the machine learning algorithm is trained to identify a number of further biological markers to assist in adjudicating the lesion.
